(19) United States
(12) Patent Application Publication
(10) Pub. No.: US 2018/0303799 A1
(43) Pub. Date: Oct. 25, 2018

(54) An Oral Preparation for the Treatment of Cardiovascular Disease and Its Preparation Method

(71) Applicant: SHENZHEN SALUBRIS PHARMACEUTICALS CO., LTD, Shenzhen, Guangdong (CN)

(72) Inventors: Ke SONG, Shenzhen (CN); Hong ZHOU, Shenzhen (CN); Guanhao Ye, Shenzhen (CN)

(21) Appl. No.: 15/768,649

(22) PCT Filed: Oct. 14, 2016
(86) PCT No.: PCT/CN2016/102132

§ 371 Date: Apr. 16, 2018

(30) Foreign Application Priority Data
Oct. 16, 2015 (CN) 201510674209.0

(51) Int. Cl. A61K 31/41; A61K 31/216; A61K 9/16; A61P 9/04; A61P 9/06; A61P 9/10; A61P 9/12; A61P 25/06; A61P 27/02

(52) U.S. Cl. CPC A61K 31/41; A61K 31/216; A61K 9/1682; A61K 9/1652; A61K 9/1641; A61P 27/02; A61P 9/04; A61P 9/06; A61P 9/10; A61P 9/12; A61P 25/06; A61K 9/1623

(57) ABSTRACT

The invention provides an oral preparation containing compound 1 used for cardiovascular disease, the oral preparation is prepared to ribbon via dry granulation process, the oral preparation comprises compound 1, filler, binder and disintegrant, and the ribbon density in process is between 0.7 and 1.5 g/cm³.

### TECHNICAL FIELD

The invention relates to the field of pharmaceutics preparation, in particular relates to an oral preparation for the treatment of cardiovascular disease and its preparation method.

### BACKGROUND ART

The trend of the risk factor of cardiovascular disease is obvious in China. With the development of social economy, the life style of people has undergone great changes, in particular, because of the aging of the population and the acceleration of urbanization process, the trend of the risk factor of cardiovascular disease is rising obviously in China, which results in the progressively increase of the number of patients with cardiovascular disease. Generally, the prevalence rate and death rate of cardiovascular disease are still going up in China, and the number of patients with cardiovascular disease will still increase rapidly over the next 10 years. At present, deaths caused by cardiovascular disease takes the first place in the total causes of death of urban and rural residents, which is 44.8% in country and 41.9% in city. The burden of cardiovascular disease is getting worse, and the cardiovascular disease has become a major public health problem (Chinese Cardiovascular Disease Report 2014).

Heart failure (HF for short) is a complex group of clinical syndromes caused by any cardiac structural or functional abnormalities resulting in impaired ventricular filling or impaired ejection. Main clinical manifestations of heart failure are dyspnea, weakness (activity tolerance is limited) and fluid retention (pulmonary congestion and peripheral edema). HF is the serious and end stage of various kinds of cardiac diseases, has high morbidity and is one of commonest cardiovascular diseases at present (Guidelines for Diagnosis and Treatment of Heart Failure in China 2014). Statistically, the number of chronic cardiac patients in China is about 4.5 million, and the prevalence rate is 0.9%, wherein the male accounts for 0.7%, and female accounts for 1.0%. According to the retrospective analysis approach by the scholars of Beijing PLA General Hospital on the inpatients with chronic heart failure for 15 years, it shows that: the 30-day mortality of the inpatients with chronic heart failure is 5.4%. Angiotensin-Converting Enzyme Inhibitors (ACEI) is proved to be the first-line drug being able to reduce the mortality of patients, as well as the drug which has the most accumulated evidences of evidence-based medicines, and is recognized as the initial therapy drug for treating HF. Enalapril is one of ACEIs for the clinical treatment of HF, but there are many adverse reactions occurring during the clinical use of ACEIs, such as cough, angioedema, hyperkalemia, deterioration of renal function and the like, and its clinical therapeutic effect needs to be further improved.

Patent WO02007056546 discloses a compound 1 for treating HF, the compound is supermolecular complex (compound) formed of compound 2 and compound 3 via covalent bonding and has the dual-acting of blocking angiotensin receptor and inhibiting neutral endopeptidase. The clinical experiment result shows that compared with the group of treatment by Enalapril, the compound 1 reduces the hospitalization rate of the subjects by 21%, reduces the symptoms and physical limitations of heart failure and is better than Enalapril in the aspect of reducing the mortality and hospitalization rate of patients with heart failure (N Engl J Med, 2014, 371(1): 993-1004). According to the authority forecast, its peak global annual sales is expected to reach 5-10 billions dollars. It can be seen that compound 1 is a kind of HF therapy drug with great market potential, and the product has been approved by FDA in the second half of 2015

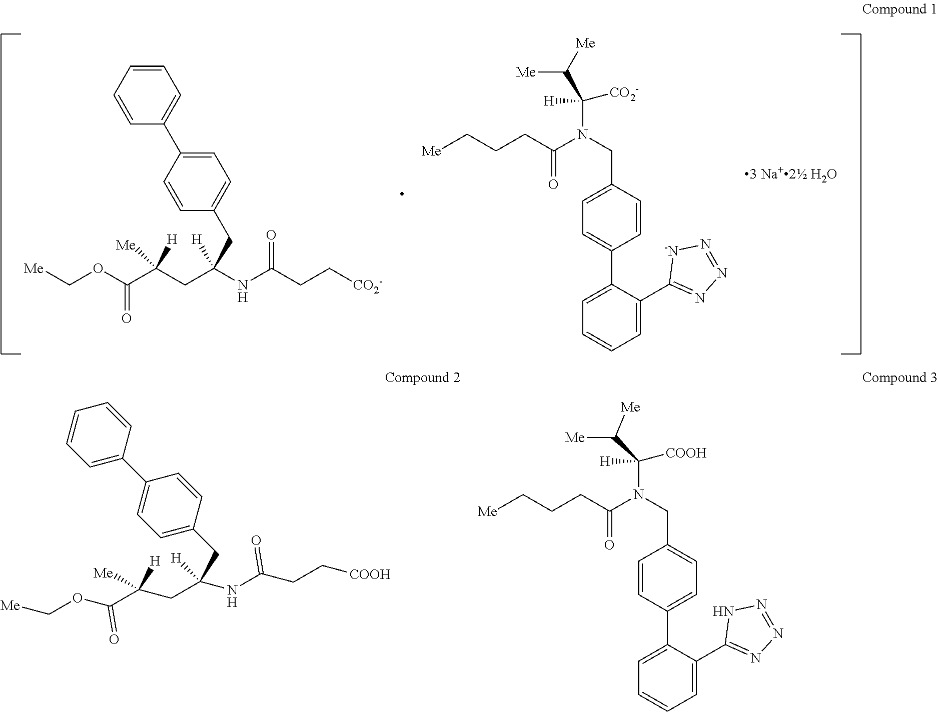

The compound 1 preparation is prepared via dry granule process in the available technologies. Patent WO2009061713 discloses an oral preparation containing compound 1, which is prepared by dry granulation process, and the obtained preparation can realize more than 70% dissolution within 20 min, but the process has neither disclosed specific technical parameters (such as granulation process, ribbon density and the like), nor implied the control of which specific technical parameters is advantageous to improve the technological level; in practice, for the preparation of compound 1, the realization of equally qualified preparation dissolution rate can correspond to different preparation processes. Different preparation processes can prepare and obtain the preparation with qualified dissolving performance, but it is still very necessary to optimize the technological level, so as to achieve the aims of simplifying process, improving product quality (such as percent of pass and the like) and other purposes.

Therefore, the technical problems to be solved by the available technologies are optimizing on the basis of available technologies, further realizing the simplification of preparation process and improving the production quality.

### CONTENT OF THE INVENTION

The first objective of the invention is to overcome the disadvantages of the available technologies and provides an oral preparation containing compound 1, which is prepared by dry granulation process, the invention improves the level of dry granulation and optimizes the preparation process by controlling the ribbon density within specific range in the dry granulation process, and the dissolution performance of the preparation also meets the clinical medication requirements of compound 1.

The above beneficial effects of the invention are realized by the following technical solutions: An oral preparation containing compound 1, prepared by dry granulation process, characterized in that the ribbon of the dry granulation process contains compound 1, filler, binder and disintegrant, and the ribbon density is 0.7-1.5 g/cm³.

The ribbon in the invention is obtained by mixing and compacting the powder of compound 1 and excipients through dry granulation, subsequently obtaining granules by smashing and granulating; the ribbon density is detected by gravimetric method, which obtains the ribbon density by measuring the length, width, thickness and weight of uniformly rectangular ribbon and then by calculating mass/volume. The ribbon density may affect the property of obtained granules and have influence on both fluidity and particle size distribution of granules and will further affect and result the tabletting process and tablet quality. During the optimization of the process preparing oral preparation of compound 1, we found that for the oral preparation of compound 1, the ribbon density has the maximum influence on the process and the quality of preparation; when the ribbon density is controlled within 0.7-1.5 g/cm³ during the dry granulation process, the solid oral preparation helps to realize the process optimization. In particular, when the ribbon density is lower, the obtained granules have small particle size and poor fluidity. On the one hand, it needs more time to mix the granules with the extra-granular excipients to achieve a certain degree of mixing uniformity, on the other hand, the poor fluidity may also influence the tabletting process, which results in the fluctuating tablet-weight of the obtained tablets and influences the product quality; when the ribbon density is higher, it will get the granules with wider particle size distribution, prolong the time for mixing the granules with extra-granular excipients and will also influence the tablet-weight variation in the process of tabletting, which results in great fluctuation of product quality, or even influences the rate of finished products. During the research and development process, we are surprising to find that for the same formula with other process conditions unchanged, when the ribbon density is 0.7-1.5 g/cm³, the granules of compound 1 obtained by smashing and granulating can realize better granule obtaining, specifically, the obtained granules have the angle of repose lower than 40°, and the obtained granules are uniform. Narrow particle size distribution can be realized without further granulating, and the better effect of mixing the granules with the extra-granular excipients can be achieved faster in the subsequent preparation process, that is to say, the mixing uniformity (RSD) is controlled within the better value less than 5%, which optimizes the preparation process while ensuring the quality stability of obtained preparation, preferably the ribbon density is 0.8-1.4 g/cm³, more preferably 0.9-1.2 g/cm³.

The compound 1 is preferably of sole crystal form, the sole crystal form is preferably the crystal form of compound 1 disclosed in patent WO2007056546, and in particular, the x-ray powder diffraction pattern of the crystal form contains the following lattice plane interval: 21.2 (s), 17.0 (w), 7.1 (s), 5.2 (w), 4.7 (w, 4.6 (w), 4.2 (w), 3.5 (w) and 3.3 (w).

The formula of ribbon preparing the said oral preparation contributes to realize the above improved beneficial effects.

Specifically, the filler is one, or mixture of two, or mixture of more than two selected from microcrystalline cellulose, lactose, starch, pregelatinized starch, mannitol, calcium hydrophosphate and sorbitol, preferably microcrystalline cellulose, mannitol, calcium hydrophosphate and sorbitol; when the part by mass of compound 1 is 1, the dosage part of the filler is 0.2-0.8, preferably 0.3-0.7, more preferably 0.4-0.6.

The disintegrant is one, or mixture of two, or mixture of more than two selected from crospovidone, sodium carboxymethyl starch, crosslinked sodium carboxymethyl cellulose and calcium carboxymethyl cellulose, preferably crosslinked sodium carboxymethyl cellulose and crospovidone, when the part by mass of compound 1 is 1, the dosage part of the disintegrant is 0.03-0.3, preferably 0.04-0.2, more preferably 0.05-0.15.

The binder is one, or mixture of two, or mixture of more than two selected from low-substituted hydroxypropyl cellulose, hydroxypropyl methylcellulose, sodium carboxymethyl cellulose, povidone and ethyl cellulose, preferably sodium carboxymethyl cellulose and low-substituted hydroxypropyl cellulose, when the part by mass of compound 1 is 1, the dosage part of the binding agent is 0.05-0.5, preferably 0.1-0.4, more preferably 0.15-0.35.

The formula of ribbon preparing the said oral preparation may further contains glidant, the glidant is one or mixture of two selected from silica and talcum powder, when the part by mass of compound 1 is 1, the dosage part of the glidant is 0.002-0.05.

The formula of ribbon preparing the said oral preparation can further contains lubricant, the lubricant is one, or mixture of two, or mixture of more than two selected from magnesium stearate, hydrogenated vegetable oil, polyethylene glycols, stearic acid, palmitic acid and carnauba wax, when the part by mass of compound 1 is 1, the dosage part of the lubricant is 0.01-0.1.

In one preferred formula for preparing ribbon in the invention, the dosages of compound 1 and excipients are as follow:

| Category | Part by mass |
|---|---|
| compound 1 | 1 |
| microcrystalline cellulose | 0.50 |
| crosslinked sodium carboxymethyl cellulose | 0.10 |
| low-substituted hydroxypropy cellulose | 0.25 |
| silica | 0.01 |
| magnesium stearate | 0.03 |

In one preferred formula for preparing ribbon in the invention, the dosages of compound 1 and excipients are as follow:

| Category | Part by mass |
|---|---|
| compound 1 | 1 |
| mannitol | 0.51 |
| crosslinked sodium carboxymethy cellulose | 0.05 |
| low-substituted hydroxypropyl cellulose | 0.25 |
| silica | 0.01 |
| magnesium stearate | 0.03 |

In one preferred formula for preparing ribbon in the invention, the dosages of compound 1 and excipients are as follow:

| Category | Part by mass |
|---|---|
| compound 1 | 1 |
| microcrystalline cellulose | 0.55 |
| crospovidone | 0.15 |
| low-substituted hydroxypropyl cellulose | 0.15 |
| silica | 0.01 |
| magnesium stearate | 0.03 |

As a whole, the above mentioned formula of preparing ribbon allows that when the ribbon density is controlled at 0.7-1.5 g/cm³, the density of obtained ribbon is moderate, the granules suitable for subsequent preparation process can be obtained after the subsequent granulating process, and the tablet weight of obtained preparation has high degree of uniformity.

The ribbon density according to the invention can be controlled by adjusting one or more than one technical parameters among roller spacing, roller speed, feeding speed and the like, so as to obtained desired ribbon density. Preferably, in production, the ribbon density is controlled by simultaneously controlling the roller spacing, the roller speed and the feeding speed. In particular, according to a preferable embodiment, it tends to obtain the ribbon with the density of 0.7-1.5 g/cm³ when controlling the roller spacing between 0.1-1.0 mm, the roller speed between 2-5 r/min, and the feeding speed between 15-25 r/min.

More specifically, several alternative preparation solutions suitable for preparing the preparation with the density within the above mentioned range are as follows:

| Roller | Roller Speed | Feeding Speed | Ribbon Density |
|---|---|---|---|
| Spacing(mm) | (r/min) | (r/min) | (g/cm³) |
| 0.7 | 4 | 24 | 0.97 |
| 0.4 | 4 | 18 | 0.84 |
| 0.2 | 2 | 22 | 1.42 |
| 0.8 | 3 | 25 | 1.19 |

The oral preparation of the compound 1 also contains extra-granular excipients which include disintegrant, lubricant and so on.

The extra-granular added disintegrant is one or mixture of two or more than two selected from crospovidone, sodium carboxymethyl starch, crosslinked sodium carboxymethyl cellulose and calcium carboxymethyl cellulose, preferably crosslinked sodium carboxymethyl cellulose and crospovidone, when the part by mass of compound 1 is 1, the dosage part of the disintegrating agent is 0.02-0.2, preferably 0.03-0.15.

The said extra-granular added lubricant is one, or mixture of two, or mixture of more than two selected from magnesium stearate, hydrogenated vegetable oil, polyethylene glycols, stearic acid, palmitic acid and carnauba wax, when the part by mass of compound 1 is 1, the dosage part of the lubricant is 0.01-0.1.

Because the ribbon density is controlled within specific range in the previous step of ribbon compacting, the obtained granules are uniform with good fluidity, have narrow particle size distribution and can be mixed with the extra-granular excipients rapidly and uniformly, which helps to shorten the process time, improve mixing effect and keep the uniform mixing state in subsequent preparation process.

A preferable formula of the oral preparation of compound 1 according to the invention is as follows:

| Item | Category | Part by mass |
|---|---|---|
| Intra-granular | compound 1 | 1 |
| agents | microcrystalline cellulose | 0.50 |
| | crosslinked sodium | 0.10 |
| | carboxymethyl cellulose | |
| | low-substituted | 0.25 |
| | hydroxypropyl cellulose | |
| | silica | 0.01 |
| | magnesium stearate | 0.03 |
| Extra-granular | crosslinked sodium | 0.08 |
| excipients | carboxymethyl cellulose | |
| | magnesium stearate | 0.03 |

A preferable formula of the oral preparation of compound 1 according to the invention is as follows:

| Item | Category | Part by mass |
|---|---|---|
| Intra-granular | compound 1 | 1 |
| agents | mannitol | 0.51 |
| | crosslinked sodium | 0.05 |
| | carboxymethyl cellulose | |
| | low-substituted | 0.25 |
| | hydroxypropyl cellulose | |
| | silica | 0.01 |
| | magnesium stearate | 0.03 |
| Extra-granular | crosslinked sodium | 0.12 |
| excipients | carboxymethyl cellulose | |
| | magnesium stearate | 0.03 |

The second objective of the invention is to provide a process of preparing the oral preparation of compound 1. The oral preparation of compound 1 is prepared by dry granulation process containing the following steps:

1) Weighing the compound 1 and excipient according to the dosage in formula;
2) Mixing the compound 1 and all intra-granular excipients to obtain total mixed powder for granules;
3) Compacting the total mixed powder for granules obtained by step 2) into ribbon with the density between 0.7-1.5 g/cm³;
4) Granulating the ribbon obtained by step 3);
5) Adding extra-granular excipients into the granules obtained by step 4), and mixing uniformly;
6) Tabletting the mixture obtained by step 5) to obtain the oral preparation of compound 1.

In the above steps, the classification, category and dosage of the excipients are all corresponding to those mentioned in the first objective of the invention.

In above steps, the compound 1 and all intra-granular excipients can be pretreated by sieving in step 2), for example passing a 40-mesh sieve; the ribbon density obtained in step 3) is preferable to be between 0.8-1.4 g/cm³, more preferably to be between 0.9-1.2 g/cm³; in step 4), it preferably uses the sieve of Ø1.2 mm-1.5 mm for granulating.

Because the ribbon density is controlled within specific range in step 3), the granules obtained after granulation by step 4) are uniform, have the angle of repose less than 40°, good fluidity and narrow particle size distribution; it can realize fast and uniform mixing with extra-granular excipients in step 5), and the obtained mixture keeps good fluidity and still keeps uniform mixing state in subsequent preparation processes.

The oral preparation of the compound 1 may also be further coated as required, the coating may be film coating, sugar coating and other ordinary coatings in the field, and the coating is made of common coating materials of the field, such as hydroxypropyl methylcellulose, powdered sugar, hydroxypropyl cellulose and so on, as well as being made of ordinary coating materials on market, such as Opadry®.

The tabletting process complies with the common knowledge of the field, the specification of the oral preparation can be any value between 10-1,000 mg, preferably, the tablet specification is 50 mg, 100 mg, 200 mg or 400 mg, the tablet hardness shall be controlled between 6-10 kgf. The third objective of the invention is the use of the oral preparation of compound 1 for preparing drugs treating cardiovascular and cerebrovascular diseases and related diseases, said diseases selected from hypertension, acute and chronic heart failure, left ventricular dysfunction, hypertrophic cardiomyopathy, diabetic cardiomyopathy, supraventricular and ventricular arrhythmias, atrial fibrillation, atrial flutter, harmful vascular remodeling, myocardial infarction and its sequelae, arteriosclerosis, unstable or stable angina, secondary aldosteronism, primary and secondary pulmonary hypertension, diabetic nephropathy, glomerulonephritis, scleroderma, glomerular sclerosis, primary nephrotic proteinuria, renal vascular hypertension, diabetic retinopathy, migraine, peripheral vascular diseases, Raynaud's disease, cavity hyperplasia, cognitive dysfunction, glaucoma and stroke. Because the ribbon density is controlled within the specific range in the preparation process, the preparation process is optimized, and the obtained oral preparation of compound 1 has higher percent of pass and higher quality with the dissolution performance meeting the requirements of clinical medication better.

Compared with the available technologies, the invention has the following technical features and advantages:

1. The invention provides an oral preparation containing compound 1, which improves the granulating level of dry granulation, optimizes the preparation process and improves the preparation quality by controlling the ribbon density within a specific range during the dry granulation process;
2. The invention provides a process of preparing the oral preparation of compound 1, the ribbon density is controlled within specific range in the dry granulation process, which allows that the granules obtained after smashing and granulating are uniform, and have the angle of repose less than 40°, good fluidity and narrow particle size distribution, the granules contain little fine powder, it can realize fast and uniform mixing with extra-granule excipients, and the obtained mixture keeps good fluidity and still keeps uniform mixing state in the subsequent preparation process, and the technical level being higher;
3. The invention provides the use of the oral preparation of compound 1 for preparing drugs treating cardiovascular and cerebrovascular diseases and related diseases, said diseases selected from hypertension, acute and chronic heart failure, left ventricular dysfunction, hypertrophic cardiomyopathy, diabetic cardiomyopathy, supraventricular and ventricular arrhythmias, atrial fibrillation, atrial flutter, harmful vascular remodeling, myocardial infarction and its sequelae, arteriosclerosis, unstable or stable angina, secondary aldosteronism, primary and secondary pulmonary hypertension, diabetic nephropathy, glomerulonephritis, scleroderma, glomerular sclerosis, primary nephrotic proteinuria, renal vascular hypertension, diabetic retinopathy, migraine, peripheral vascular diseases, Raynaud's disease, cavity hyperplasia, cognitive dysfunction, glaucoma and stroke.

### EXAMPLES

In the following, the invention was further detailed in combination with examples, but it did not limit the implementations of the invention.

In the invention the ribbon density was obtained by gravimetric method, specifically, in the process of compacting of the same batch, the ribbon density should be measured by sampling at different times, and an average value should be taken after respectively detecting and calculating each density;

The examples of the invention used the dry granulator: GL-5B dry granulator from Zhejiang Future Machinery Co., Ltd.;

The angle of repose of granule in the invention was measured by the injection method recorded in Pharmaceutics (Edition 7) published by Peoples Medical Publishing House. When the angle of repose of granule was less than 40°, the granule fluidity was more suitable for the subsequent preparation processes;

The uniformity of mixing granules with extra-granular excipients the invention was measured by the particle size distribution test method recorded in Guidelines on Drug GMP (oral solid preparation) 5.2.4 (Edition 2010). In the process of preparing compound 1, it was considered as mixing uniformly when the mixing uniformity (RSD) was controlled within 5%.

The weight variation of obtained tablets in the invention was measured by the method recorded in Appendix IA of Chinese Pharmacopeia (Edition 2010), and the maximum weight variation measured among the samples was considered as the weight variation value for such batch of tablets. For the preparation of compound 1, the tablet-weight variation was controlled within ±7.5%;

The compound 1 used in the examples of the invention was prepared by the method disclosed by example 3 of patent WO2007056546.

### Example 1

### Formula:

| | | Part by | Content |
|---|---|---|---|
| Item | Category | mass | (mg/tablet) |
| Intra-granular | compound 1 | 1 | 100 |
| agents | microcrystalline cellulose | 0.50 | 50 |
| | crosslinked sodium | 0.10 | 10 |
| | carboxymethyl cellulose | | |
| | low-substituted | 0.25 | 25 |
| | hydroxypropyl cellulose | | |
| | silica | 0.01 | 1 |
| | magnesium stearate | 0.03 | 3 |
| Extra-granular | crosslinked sodium | 0.08 | 8 |
| excipients | carboxymethyl cellulose | | |
| | magnesium stearate | 0.03 | 3 |

| | |
|---|---|
| Total | 200 |

### Preparation Method:

1. The compound 1 and intra-granular excipients was screened through 40-mesh sieve for use;
2. The compound 1 and all intra-granular excipients was mixed to obtain the total mixed powder for granules;
3. The premixed mixture was compacted by dry granulator with the roller spacing of 0.7 mm, the roller speed of 4 r/min and the feeding speed of 24 r/min to obtain the ribbon with the density of 0.97 g/cm³;
4. The ribbon was granulated through the sieve of 01.2 mm-1.5 mm to obtain granules (angle of repose being 32.1°);
5. The granules was mixed with extra-granular excipients for 10 min uniformly (RSD value being 1.2%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±2.2%.

### Example 2

### Formula:

| | | Part by | Content |
|---|---|---|---|
| Item | Category | mass | (mg/tablet) |
| Intra-granular | compound 1 | 1 | 100 |
| agents | mannitol | 0.51 | 51 |
| | crosslinked sodium | 0.05 | 5 |
| | carboxymethyl cellulose | | |
| | low-substituted | 0.25 | 25 |
| | hydroxypropyl cellulose | | |
| | silica | 0.01 | 1 |
| | magnesium stearate | 0.03 | 3 |
| Extra-granular | crosslinked sodium | 0.12 | 12 |
| excipients | carboxymethyl | | |
| | cellulose | | |
| | magnesium stearate | 0.03 | 3 |

| | |
|---|---|
| Total | 200 |

### Preparation Method:

1. The compound 1 and intra-granular excipients was screened through 40-mesh sieve for use;
2. The compound 1 and all intra-granular excipients was mixed to obtain the total mixed powder for granules;
3. The premixed mixture was compacted by dry granulator with the roller spacing of 0.8 mm, the roller speed of 3 r/min and the feeding speed of 25 r/min to obtain the ribbon with the density of 1.19 g/cm³;
4. The ribbon was granulated through the sieve of 01.2 mm-1.5 mm to obtain granules (angle of repose being 33.3°);
5. The granules was mixed with extra-granular excipients for 10 min uniformly (RSD value being 1.8%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±2.1%.

### Example 3

### Formula:

| | | Part by | Content |
|---|---|---|---|
| Item | Category | mass | (mg/tablet) |
| Intra-granular | compound 1 | 1 | 100 |
| agents | microcrystalline cellulose | 0.55 | 55 |
| | crospovidone | 0.15 | 15 |
| | low-substituted | 0.15 | 15 |
| | hydroxypropyl cellulose | | |
| | silica | 0.01 | 1 |
| | magnesium stearate | 0.03 | 3 |
| Extra-granular | crospovidone | 0.08 | 8 |
| excipients | magnesium stearate | 0.03 | 3 |

| | |
|---|---|
| Total | 200 |

### Preparation Method:

1. The compound 1 and intra-granular excipients was screened through 40-mesh screen for use;
2. The compound 1 and intra-granular excipients was mixed to obtain the total mixed powder for granules;
3. The premixed mixture was compacted by dry granulator with the roller spacing of 0.5 mm, the roller speed of 3 r/min and the feeding speed of 20 r/min to obtain the ribbon with the density of 1.05 g/cm³;
4. The ribbon was granulated through the sieve of 01.2 mm-1.5 mm to obtain granules (angle of repose being 30.8°);
5. The granules was mixed with extra-granular excipients for 10 min uniformly (RSD value being 1.3%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±2.4%.

### Example 4

### Formula:

| | | Part by | Content |
|---|---|---|---|
| Item | Category | mass | (mg/tablet) |
| Intra-granular | compound 1 | 1 | 100 |
| agents | sorbitol | 0.40 | 40 |
| | crosslinked sodium | 0.10 | 10 |
| | carboxymethyl | | |
| | cellulose | | |
| | low-substituted | 0.35 | 35 |
| | hydroxypropyl cellulose | | |
| | silica | 0.01 | 1 |
| | magnesium stearate | 0.03 | 3 |
| Extra-granular | crosslinked sodium | 0.08 | 8 |
| excipients | carboxymethyl | | |
| | cellulose | | |
| | magnesium stearate | 0.03 | 3 |

| | |
|---|---|
| Total | 200 |

### Preparation Method:

1. The compound 1 and intra-granular excipients was screened through 40-mesh sieve for use;
2. The compound 1 and intra-granular excipients was mixed to obtain the total mixed powder for granules;
3. The premixed mixture was compacted by dry granulator with the roller spacing of 0.2 mm, the roller speed of 2 r/min and the feeding speed of 22 r/min to obtain the ribbon with the density of 1.42 g/cm³;
4. The ribbon was granulated through the screen of 01.2 mm-1.5 mm to obtain granule (angle of repose being 33.1°);
5. The granules was mixed with extra-granular excipients for 10 min, mixing well (RSD value being 3.3%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±3.7%.

### Example 5

### Formula:

| | | Part by | Content |
|---|---|---|---|
| Item | Category | mass | (mg/tablet) |
| Intra-granular | compound 1 | 1 | 100 |
| agents | microcrystalline cellulose | 0.44 | 44 |
| | crosslinked sodium | 0.10 | 10 |
| | carboxymethyl cellulose | | |
| | sodium carboxymethyl | 0.35 | 35 |
| | cellulose | | |
| | silica | 0.01 | 1 |
| | magnesium stearate | 0.03 | 3 |
| Extra-granular | crosslinked sodium | 0.04 | 4 |
| excipients | carboxymethyl cellulose | | |
| | magnesium stearate | 0.03 | 3 |

| | |
|---|---|
| Total | 200 |

### Preparation Method:

1. The compound 1 and intra-granular excipients was screened through 40-mesh sieve for use;
2. The compound 1 and intra-granular excipients was mixed to obtain the total mixed powder for granules;
3. The premixed mixture was compacted by dry granulator with the roller spacing of 0.4 mm, the roller speed of 4 r/min and the feeding speed of 18 r/min to obtain ribbon with the density of 0.84 g/cm³;
4. The ribbon was granulated through the screen of Ø1.2 mm-1.5 mm to obtain granule (angle of repose being 35.2°);
5. The granules was mixed with extra-granular excipients for 10 min, mixing well (RSD value being 2.5%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±3.0%.

### Example 6

### Formula:

| | | Part by | Content |
|---|---|---|---|
| Item | Category | mass | (mg/tablet) |
| Intra-granular | compound 1 | 1 | 100 |
| agents | microcrystalline cellulose | 0.51 | 51 |
| | crosslinked sodium | 0.10 | 10 |
| | carboxymethyl cellulose | | |
| | low-substituted | 0.25 | 25 |
| | hydroxypropyl cellulose | | |
| | magnesium stearate | 0.03 | 3 |
| Extra-granular | sodium crosslinked | 0.08 | 8 |
| excipients | carboxymethyl cellulose | | |
| | magnesium stearate | 0.03 | 3 |

| | |
|---|---|
| Total | 200 |

### Preparation Method:

1. The compound 1 and intra-granular excipients was screened through 40-mesh screen for use;
2. The compound 1 and intra-granular excipients was mixed to obtain the total mixed powder for granules;
3. The premixed mixture was compacted by dry granulator with the roller spacing of 0.3 mm, the roller speed of 3 r/min and the feeding speed of 20 r/min to obtain the ribbon with the density of 1.27 g/cm³;
4. The ribbon was granulated through the screen of Ø1.2 mm-1.5 mm to obtain granules (angle of repose being 38.6°);
5. The granules was mixed with extra-granular excipients for 10 min, mixing well (RSD value being 2.8%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±3.4%.

### Example 7

### Formula:

| | | Part by | Content |
|---|---|---|---|
| Item | Category | mass | (mg/tablet) |
| Intra-granular | compound 1 | 1 | 100 |
| agents | microcrystalline cellulose | 0.56 | 56 |
| | crosslinked sodium | 0.10 | 10 |
| | carboxymethyl cellulose | | |
| | low-substituted | 0.25 | 25 |
| | hydroxypropyl cellulose | | |
| | silica | 0.01 | 1 |
| Extra-granular | crosslinked sodium | 0.08 | 8 |
| excipients | carboxymethyl cellulose | | |

| | |
|---|---|
| Total | 200 |

### Preparation Method:

1. The compound 1 and intra-granular excipients was screened through 40-mesh screen for use;
2. The compound 1 and intra-granular excipients was mixed to obtain the total mixed powder for granules;
3. The premixed mixture was compacted by dry granulator with the roller spacing of 0.2 mm, the roller speed of 3 r/min and the feeding speed of 24 r/min to obtain ribbon with the density of 1.46 g/cm³;
4. The ribbon was granulated through the screen of Ø1.2 mm-1.5 mm to obtain granules (angle of repose being 38.5°);
5. The granules was mixed with extra-granular excipients for 10 min, mixing well (RSD value being 2.8%) and the obtained mixture was tableted to obtain the core tablets of compound 1 (tablet hardness being 6.0-10.0 kgf);
6. The obtained core tablets was coated by Opadry coating polymer to obtain coated tablets. After detection, the weight variation of obtained tablets was ±3.7%.

### Comparison Example 1

Coated tablets were prepared by the same formula and preparation method as example 1, and the process difference only lied in that the ribbon with the density of 1.62 g/cm³ were prepared and obtained via dry granulator with the roller spacing of 0.2 mm, the roller speed of 3 r/min and the feeding speed of 28 r/min by adjusting the spacing and speed, granules (with angle of repose of) 44.4° were prepared and obtained after granulating, the granules and extra-granular excipients were mixed well for 10 min (RSD value of 4.2%), and the obtained mixture was tableted to obtain the core tablets of compound 1.

After detection, there were 7 tablets among the obtained tablets exceeding the limitation of weight variation, and the weight variation of obtained tablets was ±0.35% which is a high value within acceptable limit.

### Comparison Example 2

Coated tablets were prepared by the same formula and preparation method as example 1, and the process difference only lied in that the ribbon with the density of 0.65 g/cm³ were prepared and obtained by dry granulator with the roller spacing of 0.7 mm, the roller speed of 5 r/min and the feeding speed of 14 r/min by adjusting the spacing and speed, granules (with angle of repose of) 45.9° were prepared and obtained after granulating, the granules and the extra-granular excipients were mixed well for 10 min (RSD value of 4.7%), and the obtained mixture was tableted to obtain the core tablets of compound 1.

After detection, there were 4 tablets among the obtained tablets exceeding the limitation of weight variation, and the weight variation of obtained tablets was ±5.5% which is a high value within acceptable limit.

### Comparison Example 3

Coated tablets were prepared by the same formula as example 4 of patent WO2009061713. The ribbon with the density of 1.57 g/cm³ were prepared and obtained by dry granulator with the roller spacing of 0.2 mm, the roller speed of 3 r/min and the feeding speed of 27 r/min, granules (with angle of repose of 42.7°) were prepared and obtained after granulating, the granules and the extra-granular excipients were mixed well for 10 min (RSD value of 4.0%), and the obtained mixture was tableted to obtain the core tablets of compound 1.

After detection, there were 5 tablets among the obtained tablets exceeding the limitation of weight variation, and the weight variation of obtained tablets was ±4.9% which is a high value within acceptable limit.

### Example 8

In order to study the relation between ribbon density and dry granulation process and weight variation of obtained tablets, the formula of example 1 was used for obtaining ribbon with different densities by changing roller spacing and/or speed and/or feeding speed, detecting the angle of repose of the obtained granules after granulating, and mixing the obtained granules with the excipients for 10 min, so as to know the mixing effect of the granules and the extra-granular excipients, the result was as follows:

| Ribbon density | Angle of repose of | Mixing | Weight |
|---|---|---|---|
| (g/cm³) | granules | uniformity | variation |
| 0.62 | 45.6 | 4.7 | ±5.5 |
| 0.87 | 37.3 | 2.8 | ±3.3 |
| 1.15 | 33.6 | 2.4 | ±2.8 |
| 1.33 | 36.8 | 2.7 | ±3.1 |
| 1.45 | 39.2 | 3.3 | ±3.7 |
| 1.71 | 44.3 | 5.1 | ±5.3 |

It can be seen that for the dry granulation process, the ribbon density was related with the fluidity, mixing uniformity and tablet-weight variation of the obtained granules. Specifically, when the granule density was controlled within 0.7-1.5 g/cm³, the obtained granules had uniform particle size, narrow particle size distribution, little powder and good fluidity; it could realize better mixing effect when mixing with extra-granular excipients within the same time, the obtained mixture had good fluidity, and the weight variation of obtained tablets had better value within the range specified by the pharmacopeia; when the ribbon density was controlled with 0.9-1.2 g/cm³, the comprehensive assessment of dry granulation process was the highest.

Both the excessive high and the excessive low ribbon densities may result in wide particle size distribution, there was powder with large difference in particle size after smashing, and the granule fluidity disadvantage was obvious. Although the mixing uniformity barely met the requirement, there was still large fluctuation of tablet weight during the subsequent tabletting processes. Although the obtained preparation met the requirement of preparation, the quality was obviously worse than that obtained when the ribbon density was controlled within 0.7-1.5 g/cm³.

### Example 9

### Detection of Dissolution Rate

The dissolution rates of tablets of compound 1 obtained by examples 1-7 and comparison examples 1-2 were tested by the second method for determining the dissolution rate, namely the paddle method in the Appendix XC of Chinese Pharmacopoeia (Edition 2010), the resulted data were shown in the following table:

| Item | 15 min | 30 min | 45 min |
|---|---|---|---|
| Example 1 | 85.50 | 97.37 | 98.87 |
| Example 2 | 85.73 | 98.55 | 99.55 |
| Example 3 | 86.21 | 97.13 | 99.91 |
| Example 4 | 85.20 | 98.26 | 99.46 |
| Example 5 | 81.62 | 94.50 | 98.91 |
| Example 6 | 85.54 | 97.35 | 99.61 |
| Example 7 | 86.45 | 98.20 | 99.43 |
| Comparison Example 1 | 70.31 | 85.94 | 94.64 |
| Comparison Example 2 | 95.53 | 96.50 | 99.97 |
| Comparison Example 3 | 76.27 | 91.75 | 99.03 |

It can be seen that examples 1-7 and comparison examples 1-3 could all realize the dissolution of more than 70% within 15 min, basically meeting the requirements of clinical medication. Wherein, the dissolution curve of the tablets obtained by the comparison example 3 was approximately corresponding and fitting with FIG. 2 of patent WO2009061713.

In the subsequent tests of influence factor and stability, examples 1-7 all showed good stability and met the requirements of clinical medication.

The above examples are preferable implementations of the invention, but the implementations of the invention are not limited to the above examples, and any other alternation, modification, combination and simplification without deviating from the spiritual essential and principle of the invention are equivalent replacements and are all included in the protection scope of the invention.

1. An oral preparation containing compound 1, prepared by dry granulation process, characterized in that the ribbon of the dry granulation process contains compound 1, filler, binder and disintegrant, and the ribbon density is 0.7-1.5 g/cm³, preferably 0.8-1.4 g/cm³, more preferably 0.9-1.2 g/cm³.

2. The oral preparation containing compound 1 according to claim 1, characterized in that the filler is one, or mixture of two, or mixture of more than two selected from microcrystalline cellulose, lactose, starch, pregelatinized starch, mannitol, calcium hydrophosphate and sorbitol, when the part by mass of compound 1 is 1, the dosage part of the filler is 0.2-0.8, preferably 0.3-0.7.

3. The oral preparation containing compound 1 according to claim 1, characterized in that the disintegrant is one or mixture of two or more than two selected from crospovidone, sodium carboxymethyl starch, crosslinked sodium carboxymethyl cellulose and calcium carboxymethyl cellulose, when the part by mass of compound 1 is 1, the dosage part of the disintegrant is 0.03-0.3, preferably 0.04-0.2.

4. The oral preparation containing compound 1 according to claim 1, characterized in that the binder is one, or mixture of two, or mixture of more than two selected from low-substituted hydroxypropyl cellulose, hydroxypropyl methylcellulose, sodium carboxymethyl cellulose, povidone and ethyl cellulose, when the part by mass of compound 1 is 1, the dosage part of the binder is 0.05-0.5, preferably 0.1-0.4.

5. The oral preparation containing compound 1 according to claim 1, characterized in that when the part by mass of compound 1 is 1, the dosage part of the filler is 0.4-0.6, the dosage part of the disintegrant is preferably 0.05-0.15, and the dosage part of the binder is 0.15-0.35.

6. The oral preparation containing compound 1 according to claim 1, characterized in that the ribbon of the oral preparation further contains glidant, the glidant is one or mixture of two selected from silica and talcum powder, when the part by mass of compound 1 is 1, the dosage part of the glidant is 0.002-0.05.

7. The oral preparation containing compound 1 according to claim 1, characterized in that the ribbon of the oral preparation further contains lubricant, the lubricant is one, or mixture of two, or mixture of more than two selected from magnesium stearate, hydrogenated vegetable oil, polyethylene glycols, stearic acid, palmitic acid and carnauba wax, when the part by mass of compound 1 is 1, the dosage part of the lubricant is 0.01-0.1.

8. The oral preparation containing compound 1 according to claim 1, characterized in that the oral preparation of the compound 1 also contains extra-granular excipients which contain disintegrant, the disintegrant is one, or mixture of two, or mixture of more than two selected from crospovidone, sodium carboxymethyl starch, crosslinked sodium carboxymethyl cellulose and calcium carboxymethyl cellulose, when the part by mass of compound 1 is 1, the dosage part of the disintegrant is 0.02-0.2; said extra-granular excipients preferably contains lubricant which is one or mixture of two or more than two selected from magnesium stearate, hydrogenated vegetable oil, polyethylene glycols, stearic acid, palmitic acid and carnauba wax, when the part by mass of compound 1 is 1, the dosage part of the lubricant is 0.01-0.1.

9. The oral preparation containing compound 1 according to claim 8, characterized in that when the part by mass of compound 1 is 1, the dosage part of the disintegrant is 0.03-0.15.

10. The oral preparation containing compound 1 according to claim 1, characterized in that the x-ray powder diffraction pattern of the crystal form contains the following lattice plane interval: 21.2 (s), 17.0 (w), 7.1 (s), 5.2 (w), 4.7 (w, 4.6 (w), 4.2 (w), 3.5 (w) and 3.3 (w).

11. A preparation process for preparing the oral preparation of compound 1 according to claim 1, characterized in that the oral preparation of the compound 1 is prepared by dry granulation process, and the dry granulation process contains the following steps:
1) Weighing the compound 1 and excipient according to the dosage in formula;
2) Mixing the compound 1 and all intra-granular excipients to obtain total mixed powder for granules;
3) Compacting the total mixed powder for granules obtained by step 2) into ribbon with the density between 0.7-1.5 g/cm³;
4) Granulating the ribbon obtained by step 3);
5) Adding extra-granular excipients into the granules obtained by step 4), and mixing uniformly;
6) Tabletting the mixture obtained by step 5) to obtain the oral preparation of compound 1;
Preferably, the compound 1 and all intra-granular excipients is pretreated by screening in step 2), the ribbon density obtained in step 3) is preferable to be between 0.8-1.4 g/cm³, more preferably to be between 0.9-1.2 g/cm³, and in step 4), it uses the sieve of Ø 1.2 mm-1.5 mm for granulating.

12. Use of the oral preparation of compound 1 according to claim 1 to prepare drugs for treating cardiovascular and cerebrovascular diseases and related diseases, the cardiovascular and cerebrovascular diseases and related diseases are selected from hypertension, acute and chronic heart failure, left ventricular dysfunction, hypertrophic cardiomyopathy, diabetic cardiomyopathy, supraventricular and ventricular arrhythmias, atrial fibrillation, atrial flutter, harmful vascular remodeling, myocardial infarction and its sequelaes, arteriosclerosis, unstable or stable angina, secondary aldosteronism, primary and secondary pulmonary hypertension, diabetic nephropathy, glomerulonephritis, scleroderma, glomerular sclerosis, primary nephrotic proteinuria, renal vascular hypertension, diabetic retinopathy, migraine, peripheral vascular diseases, Raynaud's disease, cavity hyperplasia, cognitive dysfunction, glaucoma and stroke.
